(19) United States
(12) Patent Application Publication
(10) Pub. No.: US 2013/0304167 A1
(43) Pub. Date: Nov. 14, 2013

(54) Heat Patch for Pain

(71) Applicant: Tuming You, Xiamen (CN)

(72) Inventor: Tuming You, Xiamen (CN)

(21) Appl. No.: 13/552,540

(22) Filed: Jul. 18, 2012

Related U.S. Application Data
(63) Continuation-in-part of application No. 13/471,138, filed on May 14, 2012.

(51) Int. Cl. A61K 9/70; A61P 25/00; A61K 35/12; A61F 7/08; C01F 17/00

(52) U.S. Cl. 607/108; 423/263; 424/520; 424/443

(57) ABSTRACT

Provided in the present invention are a composition for ameliorating or treating back pain and methods of making and using the same.

### CROSS-REFERENCE TO RELATED APPLICATIONS

This application is a continuation-in-part of U.S. application Ser. No. 13/471,138, filed on May 14, 2012. The teaching in this application is incorporated herein in their entirety by reference.

### FIELD OF THE INVENTION

The invention is related to methods and articles for pain management or treatment.

### BACKGROUND OF THE INVENTION

Pain affects many people, including while color office workers and housewives. Types of pain include acute pain, chronic pain, and muscle strains.

Shoulder pain relates to shoulder muscle strain. Back pain (also known as dorsalgia) is pain felt in the back that usually originates from the muscles, nerves, bones, joints or other structures in the spine.

Back pain may have a sudden onset or can be a chronic pain; it can be constant or intermittent, stay in one place or radiate to other areas. It may be a dull ache, or a sharp or piercing or burning sensation. The pain may radiate into the arms and hands as well as the legs or feet, and may include symptoms other than pain, such as weakness, numbness or tingling.

Back pain is one of humanity's most frequent complaints. In the U.S., acute low back pain (also called lumbago) is the fifth most common reason for physician visits. About nine out of ten adults experience back pain at some point in their life, and five out of ten working adults have back pain every year.

The spine is a complex interconnecting network of nerves, joints, muscles, tendons and ligaments, and all are capable of producing pain. Large nerves that originate in the spine and go to the legs and arms can make pain radiate to the extremities.

Back pain can be divided anatomically: neck pain, middle back pain, lower back pain or tailbone pain.

By its duration: acute (less than 4 weeks), subacute (4-12 weeks), chronic (greater than 12 weeks).

By its cause: nonspecific back pain, back pain with radiculopathy or spinal stenosis, and back pain associated with another specific cause (such as infection or cancer). Non specific pain indicates that the cause is not known precisely but is believed to be due from the soft tissues such as muscles, fascia, and ligaments.

Back pain is classified according to etiology in mechanical or nonspecific back pain and secondary back pain. Approximately 98% of back pain patients are diagnosed with nonspecific acute back pain which has no serious underlying pathology. However, secondary back pain which is caused by an underlying condition accounts for nearly 2% of the cases. Underlying pathology in these cases may include metastatic cancer, spinal osteomyelitis and epidural abscess which account for 1% of the patients. Also, herniated disc is the most common neurologic impairment which is associated with this condition, from which 95% of disc herniations occur at the lowest two lumbar intervertebral levels. See, e.g., Vanwye, W R (2010 August). “Nonspecific low back pain: evaluation and treatment tips.”. The Journal of family practice 59 (8): 445-8.

According to traditional Chinese medicine, blocking of the free flow of human biological system happens in certain places (points) in the human body and cause diseases to occur. A particular disorder corresponds to blocking of a particular point or set of points in the biological system. Stimulating one of these points can free up the flow of biological system of human being so as to treat or ameliorate a disease arising out of blocking of such point or set of points. According to this school of medicine, a method of therapeutics—acupuncture—was developed and practiced in China for thousands of years. The oldest medical book known, written in China 4000 years ago, describes the use of acupuncture to treat medical problems. The use of the treatment spread to other Asian countries and to other regions of the world, including to Europe by the 1700s. In the United States, acupuncture has been used for about 200 years.

Acupuncture has long been successfully used in treatment of back and shoulder pain or strain (see, e.g., Joseph F. Audette, Phys Med Rehabil Clin N Am 15 (2004) 749-772). A common side effect of acupuncture is concern of infection caused by cross-contamination of acupuncture needles that may include viral or bacterial pathogens. Other concerns may arise out of patient's negative reactions against piercing of acupuncture needles or misapplication of such needles by a practitioner. Further, access to acupuncture by a practitioner is limited so as to limit the effect of acupuncture.

Therefore, there is a need for a different way of acupuncture treating or management paradigm for pain, e.g., back pain such as back shoulder strain.

The embodiments described below address the above identified needs and issues.

### SUMMARY OF THE INVENTION

In one aspect of the present invention, it is provided a composition for pain, e.g., back pain such as back shoulder strain, the composition comprises a heat generation component, the composition is formulated in a formulation for selective application to a set of acupoints or one or more acupoint(s) for back pain such as back shoulder strain (“acupoint(s) for back pain”) in a patient, and the composition generates heat upon application to the acupoint(s) for back pain for a sufficient period of time to generate an effect to ameliorate or treat back pain.

In some embodiments of the composition, the set of acupoints comprises the Jianjing acupoints of for back shoulder strain.

In some embodiments of the composition, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for back pain such as back shoulder strain.

In some embodiments of the composition, optionally in combination with any or all of the above various embodiments, the heat generation component comprises a rare earth component.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover is a plastic, rubber, or paper cover.

In another aspect of the present invention, it is provided a method of fabricating a composition for pain, e.g., back pain such as back shoulder strain, comprising providing a heat generation component, and formulating the composition in a formulation for selective application to a set of acupoints or one or more acupoint(s) for back pain such as back shoulder strain (“acupoint(s) for back pain such as back shoulder strain”) in a patient. The composition generates heat upon application to the set of acupoints or one or more acupoint(s) for back pain such as back shoulder strain for a sufficient period of time to generate an effect to ameliorate or treat back pain.

In some embodiments of the method, the set of acupoints for back pain comprises the Jianjing acupoints, and the back pain is back shoulder strain.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for back pain such as back shoulder strain.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments, the cover is a plastic, rubber, or paper cover.

In a further embodiment of the present invention, it is provided a method of ameliorating or treating a back pain, comprising applying, a composition at least once per day to a set of acupoints or one or more acupoint(s) for back pain such as back shoulder strain (“acupoint(s) for back pain”), which composition comprising a heat generation component, wherein the composition is formulated in a formulation for selective application to the set of acupoints or one or more acupoint(s) for back pain over the entire length of a prescribed treatment course, wherein the composition generates heat upon application to the set of acupoints or one or more acupoint(s) for back pain (such as back shoulder strain) for a sufficient period of time to generate an effect to ameliorate back pain.

In some embodiments of the method, the set of acupoints comprises the Jianjing acupoints.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for back pain.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

### BRIEF DESCRIPTION OF THE DRAWINGS

FIG. 1 shows Jianjing acupoints for back shoulder strain as an embodiment of the present invention.

### DESCRIPTION OF EXEMPLARY EMBODIMENTS OF THE INVENTION

In one aspect of the present invention, it is provided a composition for pain, e.g., back pain such as back shoulder strain, the composition comprises a heat generation component, the composition is formulated in a formulation for selective application to a set of acupoints or one or more acupoint(s) for back pain such as back shoulder strain (“acupoint(s) for back pain”) in a patient, and the composition generates heat upon application to the acupoint(s) for a sufficient period of time to generate an effect to ameliorate or treat back pain. As used herein, the term back pain includes the disorder as diagnosed by general medical standards and symptoms of back pain.

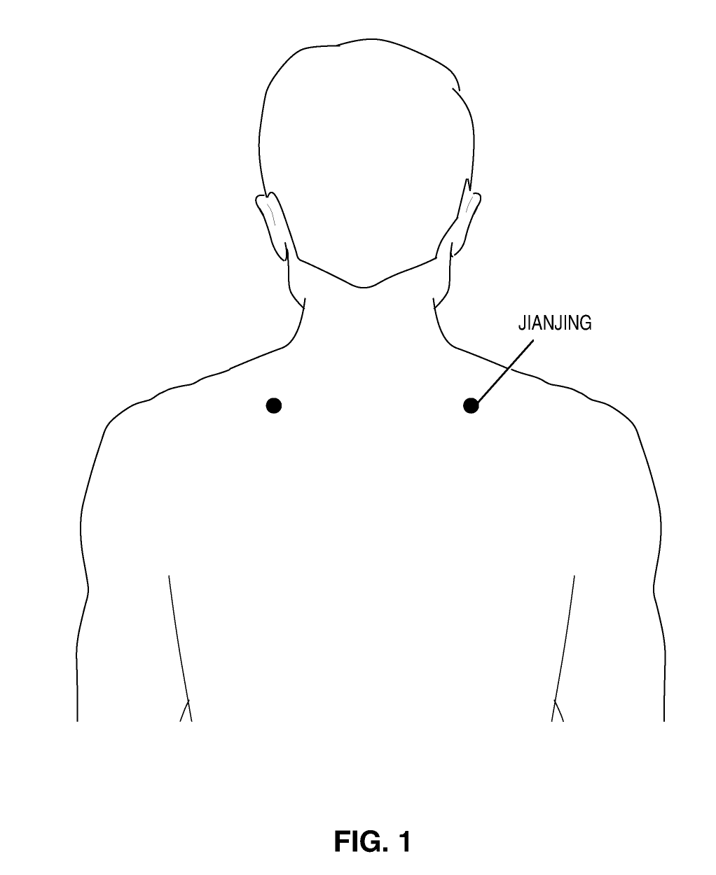

In some embodiments, the set of acupoints for pack pain comprises the Jianjing acupoints for back shoulder strain (FIG. 1).

In some embodiments, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, or 24 hrs. In some embodiments, the composition is capable of generating heat for at least 2 hours after application to the acupoint(s) for back pain such as back shoulder strain.

In some embodiments of the composition, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover can be e.g., a plastic, rubber, or paper cover.

In another aspect of the present invention, it is provided a method of fabricating a composition for pain, e.g., back pain such as back shoulder strain, comprising providing a heat generation component, and formulating the composition in a formulation for selective application to a set of acupoints or one or more acupoint(s) for back pain such as back shoulder strain (“acupoint(s) for back pain”) in a patient. The composition generates heat upon application to the set of acupoints or one or more acupoint(s) for a sufficient period of time to generate an effect to ameliorate or treat back pain.

In some embodiments of the composition, the set of acupoints comprises the Jianjing acupoints.

In some embodiments of the method, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 14 hours, or 24 hours. In some embodiments, the composition is capable of generating heat for at least 2 hours after application to the acupoint(s) for back pain.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the formulation is a patch comprising a cover. In some embodiments, the cover can be e.g., a plastic, rubber, or paper cover.

In a further embodiment of the present invention, it is provided a method, of ameliorating or treating a back pain, comprising applying a composition at least once per day to a set of acupoints or one or more acupoint(s) for back pain such as back shoulder strain (“acupoint(s) for back pain”), which composition comprising a heat generation component, wherein the composition is formulated in a formulation for selective application to the set of acupoints or one or more acupoint(s) for back pain such as back shoulder strain over the entire length of a prescribed treatment course, wherein the composition generates heat upon application to the set of acupoints or one or more acupoint(s) for a sufficient period of time to generate an effect to ameliorate back pain. In some embodiments, the back pain is back shoulder strain.

In some embodiments of the method, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 15 hours or a day. In some embodiments, the composition is capable of generating heat for at least 2 hours after application to the acupoint(s) for back pain. In some embodiments, the composition is capable of generating heat for at least 2 hours after application to the acupoint(s) for back pain.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

As used herein, the term “heat generation component” can be any component capable generating heat upon exposure to moisture or air that provides a local temperature above body temperature (e.g., 37° C.) to about 50° C. Local temperature refers to the temperature of the skin area in contact with a composition disclosed herein. Such heat generation can last at least about 10 minutes, e.g., e.g., 20 minutes, 30 minutes, 60 minutes (1 hour), 2 hours, 3 hours, 4 hours, 5 hours, 6 hours, 7 hours, 8 hours, 10 hours, 12 hours, 14 hours, or 24 hours. An example of the heat generating component is a chemical composition commonly in baby products or winter comfort products. For example, in some embodiments, the heat generation component can comprise iron powder, activated carbon, vermiculitm, and inorganic salt. In another embodiment, the heat generation component can comprise the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

As used herein, the term “a set of acupoints or one or more acupoint(s) for back pain” shall mean any set of acupoints or one or more acupoint(s) identified as acupuncture target for ameliorating or treating a back pain. In some embodiments, the set of acupoints or one or more acupoint(s) shall include the set of acupoints comprises the Jianjing acupoints (FIG. 1).

### Acupuncture and Acupoints

Acupuncture, practiced for several thousand years in China (Veith I. The yellow emperor's classic of internal medicine. Williams & Wilkins: Baltimore; 1949. pp 58-76.), is increasingly used worldwide in the treatment of many disorders. An accumulating body of evidence summarized in a NIH Consensus Statement on Acupuncture (NIH Consensus Development Panel. Acupuncture. JAMA 1998; 280: 1518-1524) confirms that acupuncture treatment has beneficial effects for conditions ranging from postoperative dental pain to chemotherapy-associated emesis. It is also effective as an adjunctive modality for joint and muscle pain, addictions, and asthma, for example.

### Meridians and Acupoints

In Traditional Chinese Medicine (TCM), life force or ‘Qi’ (‘chee’) is thought to circulate within energy pathways or ‘meridians’ longitudinally throughout the body. There are 14 major meridians, corresponding (loosely) to the Western definition of ‘organs.’ Acupoints are specific locations on the body considered to be connected to these energy meridians (NIH Consensus Development Panel. Acupuncture. JAMA 1998; 280: 1518-1524; Vickers A, Zollman C. ABC of complementary medicine: acupuncture. BMJ 1999; 319: 973-976; and Mitchell ER. Fighting drug abuse with acupuncture: the treatment that works. Pacific View Press: Berkeley, Calif.; 1995). During illness Qi is thought to be out of balance, and stimulation of acupoints corrects this imbalance. Theoretically, an ‘excess’ or ‘deficiency’ of Qi can be ‘normalized’ by the specific manner of point stimulation.

In terms of traditional medicine, it is believed that acupuncture works to alter central nervous system neurotransmitter levels by stimulating peripheral nerves at acupoints. These stimulated nerves then carry the signals centrally (Stux G, Pomeranz B. Basics of acupuncture, 4th ed. Springer-Verlag: Berlin; 1998; Mann F. Acupuncture: cure of many diseases. Butterworth-Heinemann Ltd.: Oxford; 1992), including to the spinal cord, pituitary, and midbrain. Activated centers can then release neurochemicals: endorphins, monoamines, and cortisol (Stux G, Pomeranz B. Basics of acupuncture, 4th edn. Springer-Verlag: Berlin; 1998).

### Acupoint Stimulation

A large number of acupoints (365 points have been identified in Chinese acupuncture maps) show concentrations of peripheral nerve junctions, referred to as ‘trigger points (Vickers A, Zollman C. ABC of complementary medicine: acupuncture. BMJ 1999; 319: 973-976). A variety of methods are used to stimulate acupoints (NIH Consensus Development Panel. Acupuncture. JAMA 1998; 280: 1518-1524; Stux G, Pomeranz B. Basics of acupuncture, 4th edn. Springer-Verlag: Berlin; 1998; Lao L. Acupuncture technique and devices. J Ahern Complem Med 1996; 2: 23-25). These include:
1. Needling or ‘traditional acupuncture’, in which fine stainless-steel needles are inserted through the skin to various depths, kept in place for varying lengths of time and/or further stimulated manually or electrically (‘electroacupuncture’).
2. Press needles or ‘staplepuncture’ in which short acupuncture needles are taped into place for extended periods with pressure being applied on a regular basis.
3. ‘Acupressure’ in which beads are massaged or pressed at specific locations.
4. ‘Moxibustion’ involving the application of heat, by burning small grain-sized pellets of combustible material on or near the acupoints.
5. ‘Cupping’ or using cups of various materials to create ‘negative pressure’ or a vacuum on the skin surface for increased blood circulation and point stimulation.
6. Transcutaneous electrical nerve stimulation (‘TENS’) which involves applying electrode pads to the skin surface which transmit a mild current into the acupoint.
   The teachings in the references of preceding paragraphs are incorporated herein by reference.

### Acupoints for Back Pain

In one embodiment, for providing complementary treatment of back pain using the device of invention, acupoints generally include Jianjing. These acupoints are fully described in the acupoints diagram generally available in the art. These acupoints are generally applicable to all types of back shoulder strain.

In some embodiments, the acupoints can include the above described set of acupoints and additional acupoints described in the art, such as Mingmen, Shenshu, Yaoyan, Dachangshu, Huantiao, and Weizhong, which are generally available in the art. In some other embodiments, the acupoints can further other additional acupoints, which are generally known in the art.

A number of known acupoints for back pain are described in the field. For example, Melzack, R., et al. describes quite a number of acupoints for pain, Pain, 3, p. 3-23 (1977).

### Formulations

The composition of invention can be formulated in any formulation suitable for topical, selective application to one or more acupoint(s) for back pain such as back shoulder strain. One example of the formulation is a paste. Another example is a patch. The patch can include having a cover or protective layer one side of which has a composition of invention placed thereonto. The cover can be plastic, paper, or a foil (e.g., aluminum containing foil). A thin film can be used to sandwich the composition with the cover so as to prevent the composition from exposure to moisture prior to application to acupoint(s) for back pain such as back shoulder strain of a user. A person of ordinary skill in the art can readily formulate a composition of invention into any of such formulations.

### EXAMPLES

### Example 1

### Complementary Treatment for Pain, e.g., Back Pain Such as Back Shoulder Strain

A composition disclosed herein is formulated into a patch and applied to one or any of the acupoints for pain, e.g., back pain such as back shoulder strain of a patient. The patch generates heat to cause the temperature of the one or more of the acupoints to have a temperature of about 50° C., which lasts several hours. The procedure can be repeated over a course that lasts from a week to several weeks. Improvement on patient conditions is observed.

In a particular case, a female patient that suffers back should strain used two patches on the Jianjing acupoints overnight. The unpleasant feeling of back shoulder strain disappeared (data not shown).

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

I claim:

1. A composition for pain, e.g., back pain such as back shoulder strain, comprising a heat generation component, wherein the composition is formulated in a formulation for selective application to a set of acupoints or one or more acupoint(s) for back pain (“acupoint(s) for back pain”) in a patient, wherein the composition generates heat upon application to the acupoint(s) for back pain for a sufficient period of time to generate an effect to ameliorate or treat back pain.

2. The composition of claim 1, wherein the set of acupoints comprises the Jianjing acupoints, and wherein the back pain is back shoulder strain.

3. The composition of claim 1 capable of generating heat for at least 1 hour after application to the set of acupoints or one or more acupoint(s) for back pain.

4. The composition of claim 1 capable of generating heat for at least 2 hours after application to the set of acupoints or one or more acupoint(s) for back pain.

5. The composition of claim 1, wherein the heat generation component comprises a rare earth component.

6. The composition of claim 5, wherein the heat generation component comprises a rare earth magnetic powder, saussurea in volucrata, musk, sea otter, chuanwu, caowu, lilac, menthol, meiyao, wujiapi, and maqianzhi.

7. The composition of claim 1, wherein the formulation is a patch comprising a cover.

8. The composition of claim 1, wherein the cover is a plastic, rubber, or paper cover.

9. A method of fabricating a composition for pain, e.g., back pain such as back shoulder strain, comprising providing a heat generation component, and formulating the composition in a formulation for selective application to a set of acupoints or one or more acupoint(s) for back pain such as back shoulder strain (“acupoint(s) for back pain”) in a patient, wherein the composition generates heat upon application to the set of acupoints or one or more acupoint(s) for back pain for a sufficient period of time to generate an effect to ameliorate or treat back pain.

10. The method of claim 9, wherein the composition is capable of generating heat for at least 2 hour after application to the set of acupoints or one or more acupoint(s) for back pain.

11. The method of claim 9, wherein the set of acupoints or one or more acupoint(s) for back pain comprises Jianjing acupoints, and wherein the back pain is back shoulder strain.

12. The method of claim 9, wherein the heat generation component comprises a rare earth component.

13. The method of claim 12, wherein the heat generation component comprises a rare earth magnetic powder, saussurea in volucrata, musk, sea otter, chuanwu, caowu, lilac, menthol, meiyao, wujiapi, and maqianzhi.

14. The method of claim 9, wherein the formulation is a patch comprising a cover.

15. The method of claim 9, wherein the formulation is a patch comprising a cover which comprises paper, plastic or rubber.

16. A method of ameliorating or treating back pain, comprising:
applying a composition at least once per day to a set of acupoints or one or more acupoint(s) for back pain such as back shoulder strain (“acupoint(s) for back pain”) in a back shoulder strain patient, which composition comprising a heat generation component, wherein the composition is formulated in a formulation for selective application to the set of acupoints or one or more acupoint(s) for back pain over the entire length of a prescribed treatment course, wherein the composition generates heat upon application to the set of acupoints or one or more acupoint(s) for back pain for a sufficient period of time to generate an effect to ameliorate back pain.

17. The method of claim 16, wherein the composition is capable of generating heat for at least 2 hour after application to the set of acupoints or one or more acupoint(s) for back pain.

18. The method of claim 16, wherein the set of acupoints or one or more acupoint(s) for back pain comprises Jianjing acupoints, and wherein the back pain is back shoulder strain.

19. The method of claim 16, wherein the heat generation component comprises a rare earth component.

20. The method of claim 19, wherein the heat generation component comprises a rare earth magnetic powder, saussurea in volucrata, musk, sea otter, chuanwu, caowu, lilac, menthol, meiyao, wujiapi, and maqianzhi.

21. The method of claim 16, wherein the formulation is a patch comprising a cover.

22. The method of claim 16, wherein the cover is a plastic, rubber, or paper cover.

23. The method of claim 16, wherein the treatment course lasts for about 1 month, 3 months, 6 months, or 12 months.
